(19) United States
(12) Patent Application Publication
(10) Pub. No.: US 2013/0158585 A1
(43) Pub. Date: Jun. 20, 2013

(54) SURGICAL BLADE WITH INTEGRATED GUARD

(71) Applicant: Todd O'BRIEN, Orono, ME (US)

(72) Inventor: Todd O'BRIEN, Orono, ME (US)

(21) Appl. No.: 13/328,973

(22) Filed: Dec. 16, 2011

(51) Int. Cl. A61B 17/3211

(52) U.S. Cl. 606/172

(57) ABSTRACT

An improved surgical blade with an integrated soft tissue guard, the blade being generally a flat, rectangular section of metal including one blunt end shaped to fit into a surgical blade handle and one sharpened end for cutting tissue, with the soft tissue guard extending from the sharpened end and being a flat, blunt, tongue-shaped protrusion extending in a perpendicular orientation to the sharpened end, where the soft tissue guard provides protection to the skin when the blade is used for resection of ingrown nails.

### BACKGROUND OF THE INVENTION

1. Technical Field

The invention relates generally to the field of surgical instruments and is directed to an improved surgical blade. More specifically, the invention is directed to an improved surgical blade incorporating a soft tissue guard for use in the resection of ingrown nails.

2. Description of Prior Art

Toenail surgeries are among the most frequently performed of all office procedures. Nearly one million toenail surgeries were performed by podiatrists on the American Medicare population in 2005 alone. The most frequently utilized surgical approaches to symptomatic ingrown toenails are procedures on the nail plate and nail matrix. Resections may be achieved by sharp excision of the nail matrix along with partial or total nail plate avulsion.

The traditional surgical blade used for excision of the nail matrix is a miniature surgical blade, commonly known as a beaver blade. A beaver blade is a flat, rectangular section of metal including one blunt end shaped to fit into a surgical blade handle, such as a scalpel handle, and one sharpened end for cutting tissue. After being mounted onto a surgical blade handle, a beaver blade, particularly those designated by the numbers 6100 and 6200, is advanced from the distal aspect of the involved nail plate proximally, thereby resecting the offending nail border. During advancement of the blade, however, the skin under the nail is often inadvertently lacerated by the lower edge of the sharpened end of the beaver blade. Any improvement in design over the beaver blade which obviates unnecessary soft tissue trauma would be desirable.

The present invention therefore has been developed with the aim of reducing the incidence of iatrogenic injuries.

It is therefore an objective of the present invention to provide an improved surgical blade that reduces the incidence of iatrogenic injuries.

It is a further objective of the present invention to provide an improved surgical blade that contains an integrated soft tissue guard to reduce the incidence of iatrogenic injuries.

It is a further objective of the present invention to provide an improved surgical blade that can be inserted into and used with a standard surgical blade handle.

It is a further objective of the present invention to provide an improved surgical blade that can be sterilized for reuse.

Other objectives of the present invention will be readily apparent from the description that follows.

### SUMMARY OF THE INVENTION

The present invention discloses a miniature surgical blade, commonly known as a beaver blade, which has been modified by the incorporation of a soft tissue guard. In one embodiment the invention describes a modified number 6100 blade incorporating a soft tissue guard; that is, a planar flange extending from the undersurface of the cutting edge of the blade. The flange protects the skin from laceration as the blade is advanced through the offending nail plate.

In another aspect of the present invention, the soft tissue guard is separately attached to the underside of the blade. In yet another aspect of the present invention, the soft tissue guard is formed together with the blade of a monolithic piece of surgical steel. The blade itself, moreover, may be symmetrical or oriented either with a left-hand bias or a right-hand bias.

Other features and advantages of the invention are described below.

### DESCRIPTION OF THE DRAWINGS

FIG. 1 is perspective side view of one embodiment of the present invention.

FIG. 2A is a plan side view of one embodiment of the present invention.

FIG. 2B is a plan top view of the embodiment of the present invention shown in FIG. 2A.

FIG. 2C is a plan side view of another embodiment of the present invention.

FIG. 3A is a perspective side view of the front end of the blade body of the present invention.

FIG. 3B is a plan top view of the front end of an embodiment of the present invention in which the cutting portion is angled to the right. Dotted lines are used to show angles.

FIG. 3C is a plan top view of the front end of an embodiment of the present invention in which the cutting portion is angled to the left. Dotted lines are used to show angles.

FIG. 4A is a plan side view of the front portion of a surgical blade handle.

FIG. 4B is a plan front view of the surgical blade handle shown in FIG. 4A.

FIG. 4C is a plan side view of the rear end of the blade body of the present invention.

FIG. 4D is a plan side view of the surgical blade of the present invention inserted into the surgical blade holder shown in FIGS. 4A and 4B. Ghost lines are used to show that portion of the surgical blade that would otherwise be obscured.

FIG. 5A is a plan side view of an embodiment of the present invention showing the surgical blade being used to excise a portion of a toenail. Ghost lines are used to show that portion of the surgical blade that would otherwise be obscured.

FIG. 5B is a plan top view of the embodiment of the present invention shown in FIG. 5A. Ghost lines are used to show that portion of the surgical blade that would otherwise be obscured.

### DESCRIPTION OF THE PREFERRED EMBODIMENT OF THE INVENTION

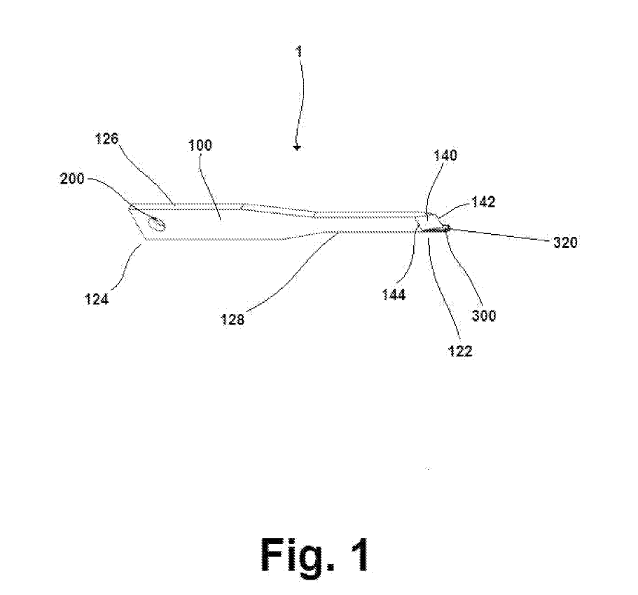

The present invention discloses an improved miniature surgical blade 1 which is generally a flat, rectangular section of metal (the blade body 100) including one blunt end (rear end 124) shaped to fit into a surgical blade handle 10 (the attachment means 200) and one sharpened end (front end 122) for cutting tissue (the cutting portion 140). The sharpened end further comprises a flat, blunt, tongue-shaped protrusion (the soft tissue guard 300) extending from the front end 122 of the surgical blade 1 in a perpendicular orientation to the sharpened end. See FIG. 1.

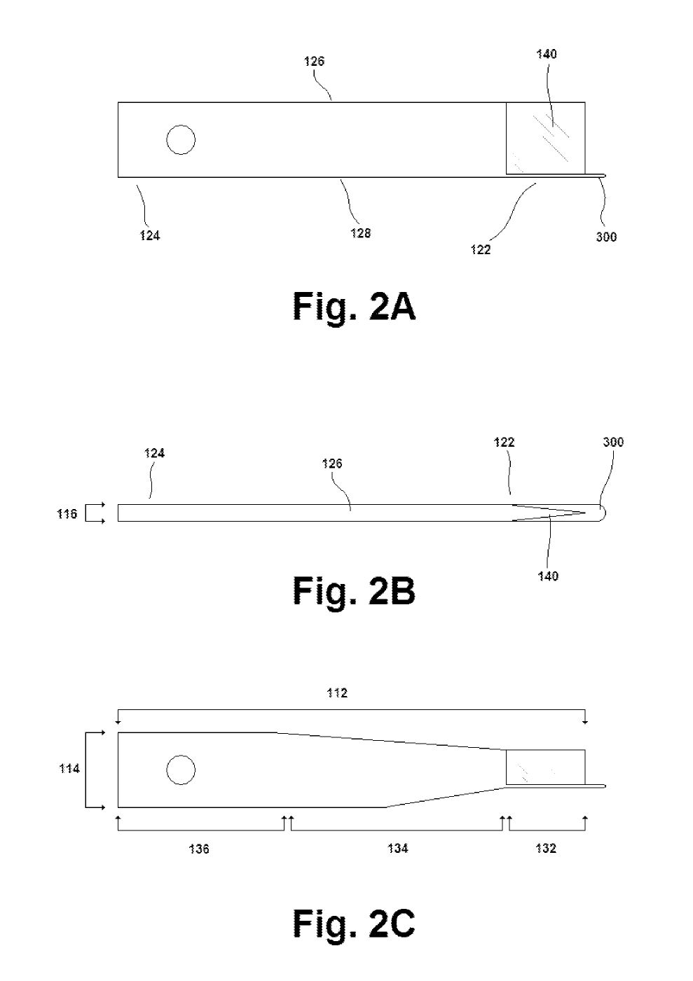

In the present invention, the blade body 100 is elongate, planar, and substantially rigid. It has a front end 122, a rear end 124, a top edge 126, a bottom edge 128, and a thickness 116. See FIG. 2A. The length 112 of the blade body 100 is greater than the height 114 of the blade body 100, and the height 114 of the blade body 100 is greater than the thickness 116 of the blade body 100. See FIGS. 2B and 2C. In the preferred embodiments, the overall length 112 of the blade body 100 is between 1.0 inches and 1.5 inches, the height 114 of the blade body 100 is between 0.04 inches and 0.15 inches, and the thickness 116 of the blade body 100 is between 0.02 inches and 0.03 inches. The blade body 100 may be manufactured of any suitable material which allows the blade body 100 to be substantially rigid, to be able to hold a sharpened edge, and to be durable. The blade body 100 should further be able to be sterilized. In the preferred embodiment the blade body 100 is manufactured of surgical steel, although other suitable materials are also contemplated.

While in one embodiment the blade body 100 is substantially rectangular, see

FIG. 2A, in other embodiments the blade body 100 may have a more complex shape, see FIG. 2C. In such embodiments, the blade body 100 may have a front portion 132 and a rear portion 136, and a middle portion 134 which is located between the front and rear portions 132,136. The shape of the rear portion 136 may be substantially rectangular and the shape of the front portion 132 may be substantially rectangular, with the height 114 of the rear portion 136 of the blade body 100 being greater than the height 114 of the front portion 132 of the blade body 100. In such configurations the middle portion 134 may be a substantially an irregular quadrilateral, with the height 114 of the middle portion 134 tapering from the rear portion 136 of the blade body 100 to the front portion 132 of the blade body 100. The top edge 126 of the middle portion 134 may comprise a single planar segment or multiple segments angled with respect to adjacent segments; similarly, the bottom edge 128 of the middle portion 134 may comprise a single planar segment or multiple segments angled with respect to adjacent segments. In the most preferred embodiment the top edge 126 of the middle portion 134 comprises a single planar segment angled downwards towards the front portion 132, while the bottom edge 128 of the middle portion 134 comprises two segments, with the segment proximate to the rear portion 136 being coplanar with the bottom edge 128 of the rear portion 136 and the segment proximate to the front portion 132 angled upwards. Other shapes for the rear, middle, and front portions 136,134,132 of the blade body 100 are also contemplated.

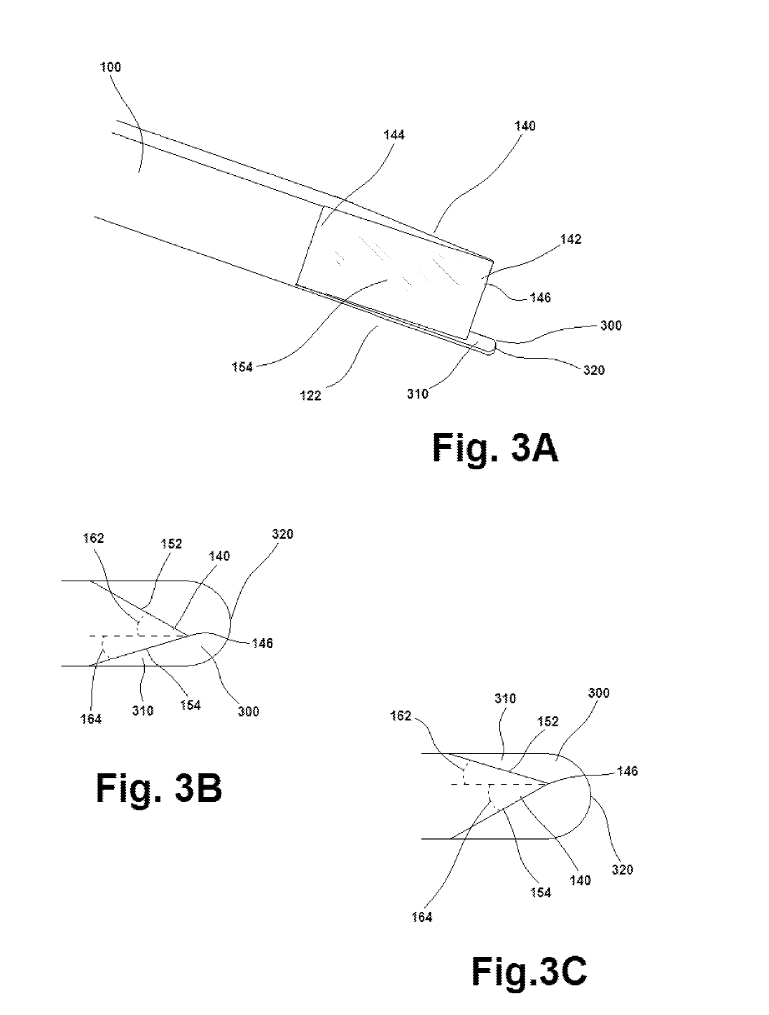

The cutting portion 140 of the improved surgical blade 1 is formed into the front end 122 of the blade body 100. See FIG. 3A. The cutting portion 140 has an anterior end 142 and a posterior end 144, with the thickness 116 of the blade body 100 tapering from the posterior end 144 of the cutting portion 140 to the anterior end 142 of the cutting portion 140, whereby the anterior end 142 of the cutting portion 140 forms a sharp leading edge 146. The cutting portion 140 spans substantially the entire height 114 of the front end 122 of the blade body 100. The left face 152 of the cutting portion 140 is substantially planar, as is the right face 154. The left and right faces 152,154 of the cutting portion 140 are separated from each other by the thickness 116 of the blade body 100 at the posterior end 144 of the cutting portion 140 and meet at and form the leading edge 146 of the cutting portion 140 at its anterior end 142. The left face 152 of the cutting portion 140 should have a width, front to back, of between 0.07 inches and 0.09 inches and the right face 154 of the cutting portion 140 should have a width of between 0.07 inches and 0.09 inches. In the preferred embodiment the left face 152 of the cutting portion 140 is oriented at a first angle 162 to the longitudinal axis of the blade body 100, and the right face 154 of the cutting portion 140 is oriented at a second angle 164 to the longitudinal axis, with the first angle 162 being substantially equal to the second angle 164. In this configuration the leading edge 146 of the cutting portion 140 is centered within the thickness 116 of the blade body 100. In the most preferred embodiments the sum of the first and second angles 162,164 is between fifteen (15) and twenty (20) degrees. Other configurations of the cutting portion 140 are also contemplated, whereby the first and second angles 162,164 differ, thereby offsetting the leading edge 146 of the cutting portion 140, allowing for left-handed and right-handed orientations of the improved surgical blade 1. See FIGS. 3B and 3C. In such embodiments the widths of the first and second faces of the cutting portion 140 will differ.

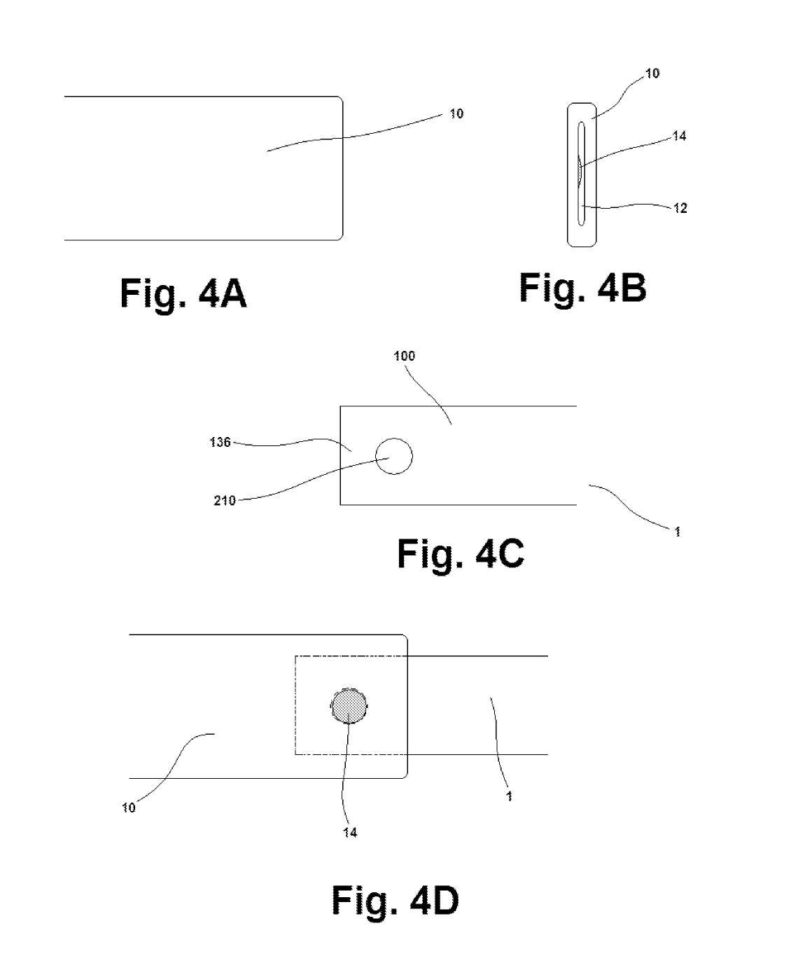

The attachment means 200 of the present invention is located proximate to the rear end 124 of the blade body 100. See FIG. 4C. It must be suitably adapted to allow the improved surgical blade 1 to be attached to a surgical blade handle 10. In one embodiment the attachment means 200 comprises the rear portion 136 of the blade body 100 and an aperture 210 formed into and through the rear portion 136 of the blade body 100. The rear portion 136 of the blade body 100 is suitably adapted to fit into a receiving portion 12 of the surgical blade handle 10, and a retaining member 14 integrated with the surgical blade handle 10 is adapted to be placed into and through the aperture 210, thereby attaching the surgical blade 1 to the surgical blade handle 10. See FIGS. 4A through 4D. The aperture 210, which may be substantially circular, may be substantially centered within the rear portion 136 of the blade body 100 between the top edge 126 of the blade body 100 and the bottom edge 128 of the blade body 100. In one embodiment the retaining member 14 is a detent ball offset by a biasing member; the detent ball is configured to fit within the aperture 210 of the surgical blade 1 and is held in place by the biasing member, in a manner as is well known in the art. Other configurations of attachment means 200 as are known in the art are also contemplated by the present invention.

The soft tissue guard 300 of the improved surgical blade 1 comprises an elongate, planar, substantially rigid, substantially rectangular flange (the main body 310). See FIG. 3A. It is located adjacent to the bottom edge 128 of the blade body 100 proximate to the front end 122 of the blade body 100. The width of the main body 310 is substantially the same as the thickness 116 of the blade body 100 at the posterior end 144 of the cutting portion 140. In the preferred embodiment the soft tissue guard 300 has a thickness of between 0.005 inches and 0.015 inches. The forward edge 320 of the main body 310 of the soft tissue guard 300 extends beyond the leading edge 146 of the cutting portion 140, such that the main body 310 of the soft tissue guard 300 is located below and adjacent to the entirety of the cutting portion 140 of the blade body 100, with the leading edge 146 of the cutting portion 140 oriented substantially perpendicular to the main body 310. In the preferred embodiment the forward edge 320 of the main body 310 of the soft tissue guard 300 should extend beyond the leading edge 146 of the cutting portion 140 by between 0.02 inches and 0.03 inches. In the preferred embodiment, the forward edge 320 of the soft tissue guard 300 is rounded. In another embodiment the forward edge 320 of the soft tissue guard 300 is tapered such that it has a greater thickness in the portion located directly under the cutting portion 140 of the blade body 100 and is thinner at the forward edge 320.

The soft tissue guard 300 may be manufactured of any suitable material. In preferred embodiments it is manufactured out of the same material as the blade body 100. In the most preferred embodiment it is manufactured out of surgical steel. In one embodiment the soft tissue guard 300 is manufactured from a separate piece of material and is thereafter attached to the bottom edge 128 of the blade body 100. It may be attached by any known means, such as fasteners, welds, adhesives, and the like. In other embodiments the soft tissue guard 300 is formed from the same piece of material as the blade body 100, resulting in a monolithic structure. To achieve this embodiment the left and right faces 152,154 of the cutting portion 140 may be formed by grinding down the sides of the front end 122 of the blade body 100, while leaving a thin portion at the bottom edge 128 of the blade body 100 untouched.

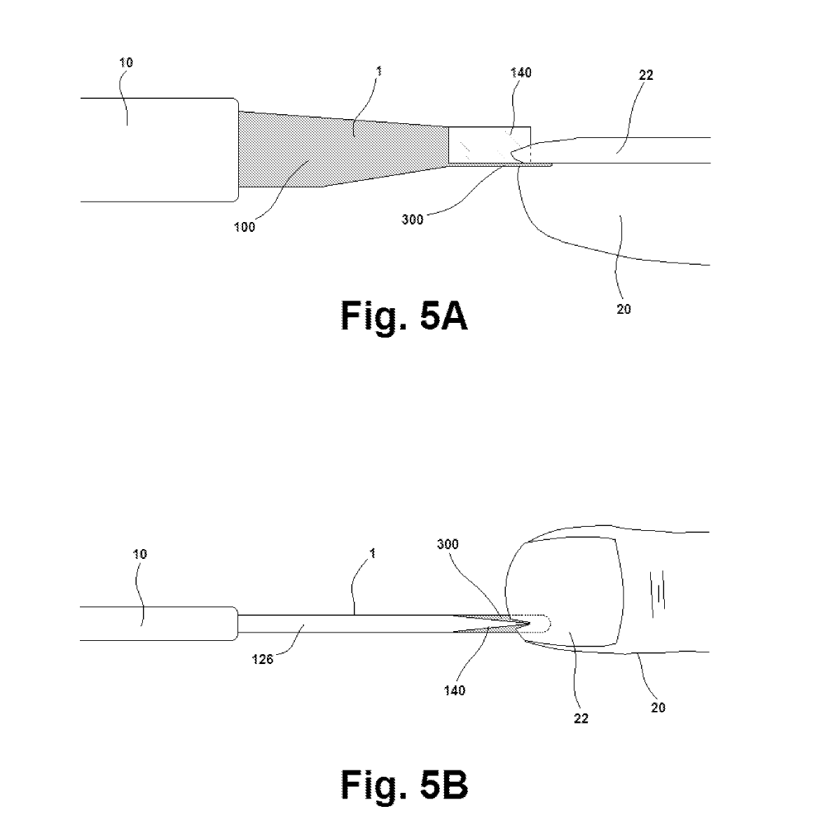

The surgical blade 1 of the present invention may be used in various surgical applications. In one application, it is used to resect ingrown toenails 22. See FIGS. 5A and 5B. The soft tissue guard 300 is inserted between the toe 20 and the toenail 22; as the surgical blade 1 is pushed forward, the leading edge 146 of the cutting portion 140 of the surgical blade 1 cuts through a portion of the toenail 22, while the soft tissue guard 300 prevents the soft tissues of the toe 20 located under the toenail 22 from being cut. The surgical blade 1 may also be used for resecting fingernails, in substantially the same manner as described herein.

What has been described and illustrated herein is a preferred embodiment of the invention along with some it its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect. Modifications and variations can be made to the disclosed embodiments of the present invention without departing from the subject or spirit thereof as defined in the following claims. Other embodiments not specifically set forth herein are also within the scope of the following claims.

I claim:

1. An improved surgical blade comprising
an elongate, planar, substantially rigid blade body, said blade body having a length, a height, a thickness, a front end, a rear end, a top edge, and a bottom edge, with the length of the blade body being greater than the height of the blade body and the height of the blade body being greater than the thickness of the blade body;
a cutting portion, said cutting portion formed into the front end of the blade body, said cutting portion having an anterior end and a posterior end, with the thickness of the blade body tapering from the posterior end of the cutting portion to the anterior end of the cutting portion to form a sharp leading edge of the cutting portion, said cutting portion spanning substantially the entire height of the blade body at the front end of the blade body, with the cutting portion further comprising a substantially planar left face and a substantially planar right face, with the left and right faces of the cutting portion being separated from each other by the thickness of the blade body at the posterior end of the cutting portion and the left and right faces of the cutting portion meeting at and forming the leading edge of the cutting portion at the anterior end of the cutting portion, whereby the left face of the cutting portion is oriented at a first angle to the longitudinal axis of the blade body and the right face of the cutting portion is oriented at a second angle to the longitudinal axis;
an attachment means, said attachment means located proximate to the rear end of the blade body, said attachment means suitably adapted to allow the improved surgical blade to be attached to a surgical blade handle; and
an elongate, planar, substantially rigid soft tissue guard, said soft tissue guard being a substantially rectangular flange that is located adjacent to the bottom edge of the blade body proximate to the front end of the blade body, said soft tissue guard having a main body, a forward edge, and a width, with the width of the soft tissue guard being substantially the same as the thickness of the blade body at the posterior end of the cutting portion, and the forward edge of the soft tissue guard extending beyond the leading edge of the cutting portion, such that the main body of the soft tissue guard is located below and adjacent to the entirety of the cutting portion of the blade body;
whereby the leading edge of the cutting portion is oriented substantially perpendicular to the main body of the soft tissue guard.

2. The improved surgical blade of claim 1 wherein the blade body is manufactured of surgical steel.

3. The improved surgical blade of claim 1 wherein the blade body is substantially rectangular.

4. The improved surgical blade of claim 1 wherein
the blade body has a front portion which encompasses the front end of the blade body, a rear portion which encompasses the rear end of the blade body, and a middle portion which is located between the front portion and the rear portion,
whereby the height of the rear portion of the blade body is greater than the height of the front portion of the blade body, with the height of the middle portion of the blade body tapering from the rear portion of the blade body to the front portion of the blade body.

5. The improved surgical blade of claim 4 wherein
the top edge of the rear portion of the blade body is substantially parallel to the bottom edge of the rear portion of the blade body, and
the top edge of the front portion of the blade body is substantially parallel to the bottom edge of the front portion of the blade body.

6. The improved surgical blade of claim 1 wherein the first angle is substantially equal to the second angle.

7. The improved surgical blade of claim 1 wherein the first angle differs from the second angle.

8. The improved surgical blade of claim 1 wherein the sum of the first angle and the second angle is between fifteen and twenty degrees.

9. The improved surgical blade of claim 1 wherein
the blade body has a rear portion which encompasses the rear end of the blade body, and
the attachment means comprises the rear portion of the blade body and an aperture formed into and through the rear portion of the blade body,
whereby the rear portion of the blade body is suitably adapted to fit into a receiving portion of the surgical blade handle and a retaining member integrated with the surgical blade handle is adapted to be placed into and through the aperture, thereby attaching the surgical blade to the surgical blade handle.

10. The improved surgical blade of claim 9 wherein the aperture is substantially centered within the rear portion of the blade body between the top edge of the blade body and the bottom edge of the blade body.

11. The improved surgical blade of claim 9 wherein the aperture is substantially circular.

12. The improved surgical blade of claim 1 wherein the soft tissue guard is manufactured of surgical steel.

13. The improved surgical blade of claim 1 wherein the forward edge of the soft tissue guard is rounded.

14. The improved surgical blade of claim 1 wherein the soft tissue guard has a thickness and the forward edge of the soft tissue guard is tapered such that the thickness of the main body of the soft tissue guard is greater than the thickness of the forward edge of the soft tissue guard.

15. The improved surgical blade of claim 1 wherein the soft tissue guard is formed into the front end of the blade body.

16. The improved surgical blade of claim 1 wherein the soft tissue guard is attached to the bottom edge of the blade body.

17. The improved surgical blade of claim 1 wherein the length of the blade body is between 1.0 inches and 1.5 inches.

18. The improved surgical blade of claim 1 wherein the height of the blade body at the front end of the blade body is between 0.04 inches and 0.08 inches.

19. The improved surgical blade of claim 1 wherein the thickness of the blade body is between 0.02 inches and 0.03 inches.

20. The improved surgical blade of claim 1 wherein the left face of the cutting portion has a width of between 0.07 inches and 0.09 inches and the right face of the cutting portion has a width of between 0.07 inches and 0.09 inches.

21. The improved surgical blade of claim 1 wherein the soft tissue guard has a thickness of between 0.005 inches and 0.015 inches.

22. The improved surgical blade of claim 1 wherein the forward edge of the soft tissue guard extends beyond the leading edge of the cutting portion by between 0.02 inches and 0.03 inches.
